(19) United States
(12) Patent Application Publication
(10) Pub. No.: US 2010/0087901 A1
(43) Pub. Date: Apr. 8, 2010

(54) Body temperature regulating device and associated method

(76) Inventor: Isreal Hernandez, Houston, TX (US)

Correspondence Address: ASHKAN NAJAFI, P.A., 6817 SOUTHPOINT PARKWAY, SUITE 2301, JACKSONVILLE, FL, 32216, US

(21) Appl. No.: 12/287,348

(22) Filed: Oct. 8, 2008

(51) Int. Cl. A61F 7/00

(52) U.S. Cl. 607/107

(57) ABSTRACT

A body temperature regulating device for distributing ambient air directly onto a user skin may include a body harness positioned on the user skin. A portable air pump for receiving and channeling ambient air may also be included. A portable air intake manifold may also be coupled to the air-pump for receiving and distributing air based on pre-defined operating parameters. A user inter face and controller may located downstream of an air intake valve for allowing a user to monitor the air intake rate and control the air distribution rate. Air may be distributed from the air intake manifold through the air distributions valves into conduit. The conduits may connect the distributions valves to each harness. A comfort valve may be located on each conduit proximate to the harness in order to allow a user to individually select and adjust the air exhaust rate into the harness.

### CROSS REFERENCE TO RELATED APPLICATIONS

This application claims the benefit of U.S. Provisional Application No. 60/965,532, filed Aug. 21, 2007, the entire disclosures of which are incorporated herein by reference.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

1. Technical Field

This invention relates to air cooling devices and, more particularly, to a body temperature regulating device for distributing ambient air directly onto a user skin.

2. Prior Art

Body overheating through vigorous participation in sports and job-related activities is a widely recognized problem which is particularly of concern in hot, humid climates where human body temperature substantially in excess of safe levels can occur. This type of body overheating can result in enforced periods of inactivity and, in many cases, may require treatment such as soaking an overheated person in cool water or immersion of the person in an ice bath. In the most extreme instances, hospitalization of the individual may result due to dehydration, heatstroke, or other serious problems associated with spending extensive periods of time in extreme temperatures.

As is well known to those familiar with football and similar rigorous sports, there are unfortunate incidents of heat-related deaths every year among groups of individuals who otherwise are recognized as exceptionally physically fit. Many other less severe forms of heat illness also occur and can result in significant health hazards to the individuals involved and attendant losses in training and playing time. These same problems are also inherent in other fields of human endeavor where the individual is exposed to a hot, humid environment. Particularly, such problems may occur during working conditions that require a worker to perform extensive manual labor in extreme temperatures.

Avoidance of body overheating stress is normally accomplished with scheduled rest periods, particularly among individuals previously recognized as susceptible to heat stress. Unfortunately, many times such rest periods may be incompatible with athletic game participation or work requirements and/or may be disregarded as unnecessary by an aggressive athlete or worker. Failure to cool down sufficiently during rest periods may consequently lead to progressive rises in body temperature which can ultimately reach dangerous levels that require off-site treatment and medical intervention. The present invention was developed to address these well recognized problems associated with heat stress in athletes as well as workers and other individuals who must perform in a hot, humid environment for an extended period of time.

In the prior art, “side-line air conditioning” or air conditioners mounted on rollers and provided with an air duct on the front thereof have been used at athletic events to cool the players. Typically, the air conditioner is rolled into a position close to the players' bench at a football game and/or rolled into the locker rooms for cooling. This type of apparatus has been used at a number of institutions including Georgia Institute of Technology and North Carolina State University. Additional prior art includes an air conditioned clear cellophane tent to accommodate multiple athletes on the side-lines at the University of Georgia in Athens, Ga. Unfortunately, these prior art examples are not designed to allow a worker or athlete to be mobile. Instead, in order to remain cool, a user must remain within a limited area.

U.S. Pat. No. 6,543,247 to Strauss discloses a waist-mounted evaporative personal cooling device designed to cool the back of a user. The device comprises a blower, a liquid reservoir, a means to deliver liquid from the reservoir in a mist of droplets into blower-forced air or directly onto the skin area to which the forced air will be directed, and a duct to guide forced air under the user's shirt or blouse and directly onto or across the skin of the user's back. The device improves on prior art coolers by delivering a powerful evaporative cooling effect directly to a user's back, while being compact, comfortably wearable, and requiring the user to do little or nothing to get its benefit. Unfortunately, this prior art reference does not provide adequate cooling action to the arms and legs of the user which also may be subjected to high levels of heat inside a work outfit.

U.S. Pat. No. 7,249,464 to Watson discloses a device with a backpack that includes a cavity extending from a top to a bottom edge wherein a fan is partially seated and blows ambient air towards the operator. A heat sink is mated to the fan, disposed anterior thereof and is situated for allowing the air to pass therethrough. A mechanism is included for supplying power to the fan and the thermal electric plate from a single power supply source to blow cool air upon a user while allowing the user to be mobile. Unfortunately, this prior art reference would not provide a high enough flow of air to reach all parts of the user's body to regulate the temperature adequately.

U.S. Pat. No. 6,192,702 to Shimogori discloses a personal cooling device that may be slung about the neck of a wearer for providing personal cooling under various conditions. The device is a light-weight air conditioner that is battery powered and may utilize one of a plurality of cooling packs, preferably a gel pack. The pack is installed within the container and a motor operated fan is adapted to blow cool air on the user. The device may be stowed about the waistband of a garment of the user when not used for cooling purposes. Unfortunately, the prior art reference would be bulky to hang from a user's neck and may distract the user from their work.

Accordingly, a need remains for a temperature regulating device in order to overcome the above-noted shortcomings. The present invention satisfies such a need by providing a device that is convenient and easy to use, lightweight yet durable in design, and designed for creating a wall of cool air between a user and his/her environment.

### BRIEF SUMMARY OF THE INVENTION

In view of the foregoing background, it is therefore an object of the present invention to provide a device for distributing ambient air directly onto a user skin. These and other objects, features, and advantages of the invention are provided by a body temperature regulating device.

A body temperature regulating device for distributing ambient air directly onto a user skin preferably includes a body harness adapted to be directly positioned on the user skin and a mechanism for automatically receiving and distributing ambient air towards the body harness based upon a plurality of pre-defined operating parameters. Allowing the user to wear the body harness directly against their skin provides for easily concealing the harness beneath the user's work clothes and non-prohibitive access for the air to reach the body. The pre-defined operating parameters may include an ambient air intake rate measured upstream of the body harness and a plurality of ambient air distribution rates measured downstream of the ambient air intake rate.

The regulating device may further include a mechanism for manually adjusting an ambient air exhaust rate leading directly into the body harness to thereby manually and independently override the pre-defined operating parameters when desired by the user. In this manner, a user may manually select the amount of air distributed to their body based on the fluctuation of exterior temperatures to regulate their body temperature accordingly.

The automatic ambient air receiving and distributing mechanism may further include a portable air-pump for receiving and channeling the ambient air. Such an air pump would preferably be light-weight and easily stored in a work truck or shop to be transported to jobs as needed. Additionally, the mechanism may include a portable air intake manifold communicatively coupled to the air-pump for receiving and distributing the ambient air based upon the pre-defined operating parameters. Further, the ambient air may be directed into the air intake manifold at an ambient air intake rate and thereafter distributed away from the air intake manifold at ambient air distribution rates respectively. The air receiving and distributing mechanism may also include each of the ambient air distribution rates traveling along mutually exclusive paths away from the air intake manifold.

The automatic ambient air receiving and distributing mechanism may further include a user interface and a controller communicatively coupled to the user interface. In addition, the mechanism preferably includes an intake valve positioned upstream of the controller for receiving the ambient air at the ambient air intake rate. Also, the mechanism may include an intake valve sensor communicatively coupled to the intake valve that is preferably situated downstream thereof. Such an intake valve sensor preferably generates and transmits a main signal to the controller that identifies a level of the ambient air intake rate.

A plurality of distribution valves positioned downstream of the controller for receiving the ambient air at the distribution rates respectively is also preferably included. Further, the mechanism may include a plurality of distribution valve sensors communicatively coupled to the distribution valves that may be situated downstream thereof respectively. Each of such distribution valve sensors preferably generates and transmits auxiliary signals to the controller that identify a corresponding level of the ambient air distribution rates respectively. Additionally, the mechanism may include a plurality of conduits connected to the intake valve and the distribution valves respectively. Each of such conduits travels along mutually exclusive paths and further may be arranged in such a manner that a separate and unique volume of the ambient air is preferably directed through the conduits during operating conditions.

The body temperature regulating device may further include a memory with programmable software instructions that cause the distribution valves to automatically toggle between open and closed positions based upon real-time fluctuations detected in the operating parameters. This frees the user's hands to complete work tasks while allowing the device regulate the air as needed. The software instructions may further include a programmable control logic algorithm preferably executing the steps of first requesting a user to designate a desired level of the ambient air intake rate via the user interface.

Secondly, the algorithm preferably requests a user to designate a desired level of the ambient air distribution rates via the user interface. Third, the algorithm may receive the main signal and learn a real-time ambient air intake rate therefrom. Next, the algorithm may receive the auxiliary signals and learn real-time ambient air distribution rates therefrom respectively.

A fourth step preferably includes determining whether one of the real-time ambient air intake and distribution rates does not equal a corresponding one of the desired ambient air intake and distribution rates levels respectively. If the fourth step is yes, then the algorithm's fifth step may include generating and transmitting a corresponding output control signal to an associated one of the intake and distribution valves to thereby automatically adjust the one real-time ambient air rate to a corresponding one of the desired ambient air intake and distribution rates respectively.

The ambient air exhaust rate adjusting mechanism may further include a comfort valve located downstream of an associated one of the conduits and located proximate to the body harness. Such a comfort valve may include a hollow chamber and an inlet port located at a first quadrant of the hollow chamber that may be in fluid communication with the one conduit for receiving the ambient air therein. The comfort valve may additionally include an outlet port located at a second quadrant of the hollow chamber that may be in fluid communication with the body harness.

Further, the comfort valve may include a spool centrally seated within the hollow chamber that may be freely rotated along a unidirectional arcuate path defined within the hollow chamber. In addition, a curvilinear first passageway may be defined about the spool and travel within the hollow chamber of the comfort valve. The comfort valve may also include an actuating block statically nested within the hollow chamber that preferably maintains intermittent engagement with the spool as the spool is rotated.

The ambient air exhaust rate adjusting mechanism further may include diametrically opposed orifices formed within an outer wall of the spool such that a linear second passageway may be formed through a center of the spool. Also, the mechanism may include first and second arms pivotally connected to the spool and radially extending out therefrom.

The first and second arms preferably maintain direct abutment with an inner surface of the hollow chamber when articulated to open positions. This operates so that the ambient air is prohibited from traveling beyond the first and second arms in the curvilinear first passageway. In addition, the spool may be manually rotated by the user to thereby cause one of the first and second arms to rub against the actuating block. The one arm may be pivotally positioned over a respective one of the orifices to thereby axially offset the linear second passageway away from the outlet port. This operates so that the ambient air is diverted away from the linear second passageway and along the curvilinear first passageway. Further, the ambient air exhaust rate is preferably at maximum and minimum levels when the linear second passageway is axially and transversely aligned with the outlet port respectively.

A further purpose of the present invention is to provide a method for distributing ambient air directly onto a user skin. Such a method preferably includes the chronological step of first providing and positioning a body harness directly on the user skin. Second, the method may include the step of automatically receiving and distributing ambient air towards the body harness based upon a plurality of pre-defined operating parameters. The pre-defined operating parameters may include an ambient air intake rate measured upstream of the body harness.

The parameters may also include a plurality of ambient air distribution rates measured downstream of the ambient air intake rate. Finally, the method may include the step of manually adjusting an ambient air exhaust rate leading directly into the body harness to thereby manually and independently override the pre-defined operating parameters when desired by the user. The ambient air exhaust rate is preferably located downstream of the ambient air distribution rates.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

It is noted the purpose of the foregoing abstract is to enable the U.S. Patent and Trademark Office and the public generally, especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE SEVERAL VIEWS OF THE DRAWING

The novel features believed to be characteristic of this invention are set forth with particularity in the appended claims. The invention itself, however, both as to its organization and method of operation, together with further objects and advantages thereof, may best be understood by reference to the following description taken in connection with the accompanying drawings in which:

FIG. 1 is a rear perspective view showing a body harness of a body temperature regulating device worn by a user, in accordance with the present invention;

FIG. 2 is front perspective view showing the body harness worn by the user;

FIG. 3 is a front perspective view of the present invention showing the body harness and the mechanism for automatically receiving and distributing ambient air toward the body harness;

FIGS. 3A and 3B are cross sectional views of the mechanism for manually adjusting the ambient air exhaust rate leading directly into the body harness, more specifically the comfort valve is shown at open and close positions respectively;

FIG. 4 is a schematic block diagram showing the interrelationship between the main components of the present invention; and

FIG. 5 is a schematic block diagram showing the interrelationship between the components of the controller.

### DETAILED DESCRIPTION OF THE INVENTION

The present invention will now be described more fully hereinafter with reference to the accompanying drawings, in which a preferred embodiment of the invention is shown. This invention may, however, be embodied in many different forms and should not be construed as limited to the embodiment set forth herein. Rather, this embodiment is provided so that this application will be thorough and complete, and will fully convey the true scope of the invention to those skilled in the art. Like numbers refer to like elements throughout the figures.

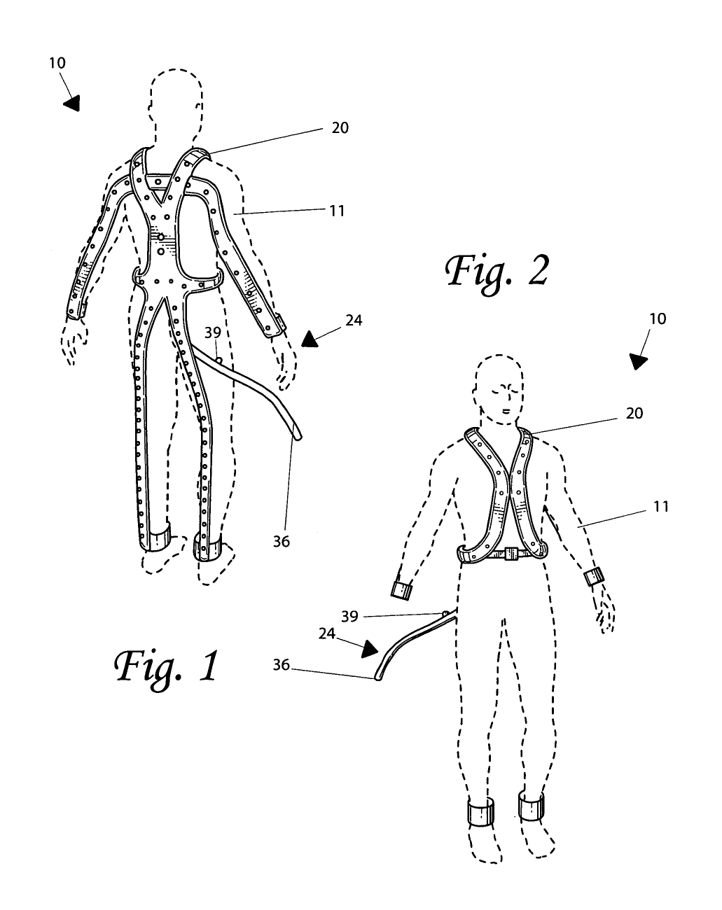

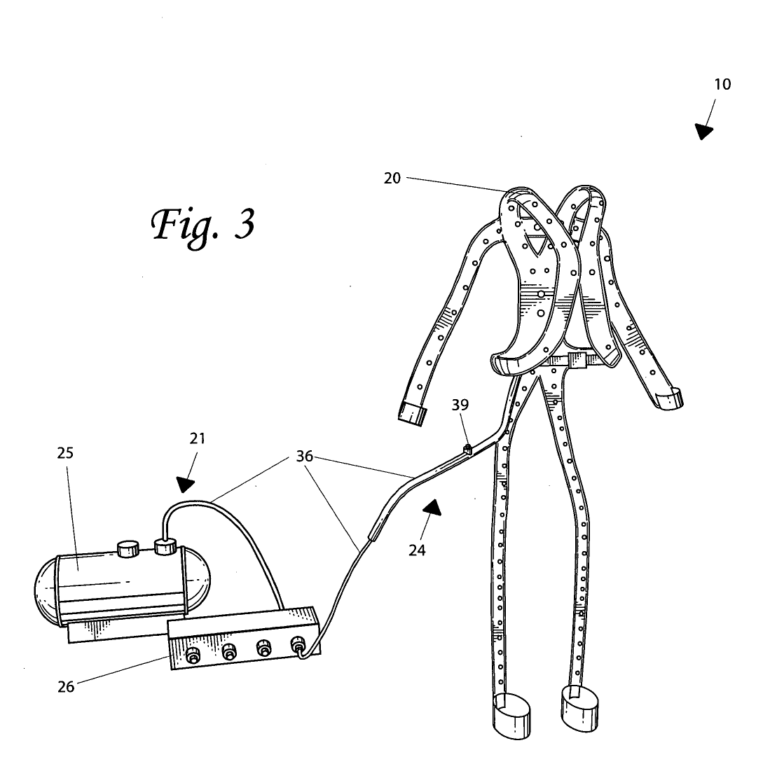

The device of this invention is referred to generally in FIGS. 1-5 by the reference numeral 10 and is intended to provide a body temperature regulating device. It should be understood that the device 10 may be used to distribute ambient air onto a user skin for many different types of purposes and should not be limited in use to the applications mentioned herein. For example, the device may be employed by a vast variety of users from athletes to industrial laborers and the like. The present invention advantageously allows a user to remain cool or warm as needed. In addition, the present invention allows a user to be mobile in their activities by connecting and disconnecting an air pump to a body harness (described hereinbelow). Such benefits overcome the prior art shortcomings as will be understood by the detailed description set forth below.

Referring initially to FIGS. 1-3, a body temperature regulating device 10 preferably includes a body harness 20 adapted to be directly positioned on the user skin 11 and a mechanism 21 for automatically receiving and distributing ambient air 12 towards the body harness 20 based upon a plurality of pre-defined operating parameters. Allowing the user to wear the body harness 20 directly against their skin 11 provides for easy concealment of the harness 20 beneath the user's clothing and generates non-prohibitive access for the air 12 to reach the body.

The pre-defined operating parameters may include an ambient air intake rate 22 measured upstream of the body harness 20 and a plurality of ambient air distribution rates 23 measured downstream of the ambient air intake rate. The device 10 may further include a mechanism 24 for manually adjusting an ambient air exhaust rate leading directly into the body harness 20 to thereby manually and independently override the pre-defined operating parameters when desired by the user. In this manner, a user may manually select the amount of air 12 distributed to their body based on the fluctuation of exterior temperatures in order to regulate their body temperature accordingly. This provides a benefit wherein the user may override the current air exhaust rate 56 when engaging in increased physical activity and thereby combating production of additional body heat.

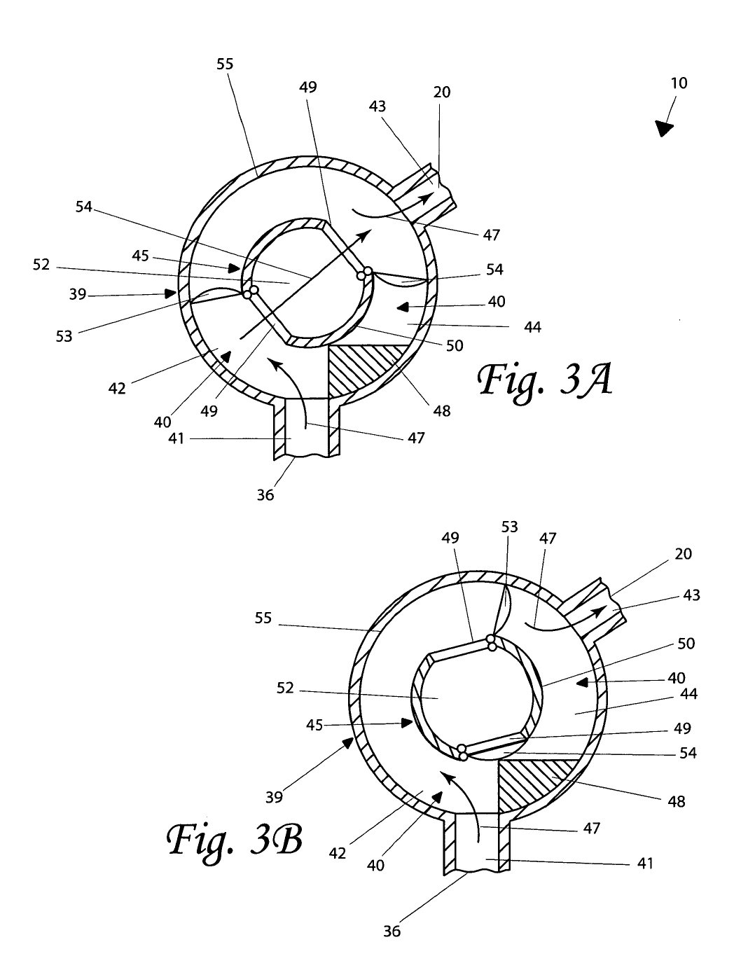

Referring to FIGS. 3-5, the automatic ambient air receiving and distributing mechanism 21 may further include a portable air-pump 25 for receiving and channeling the ambient air 12. Such an air pump 25 may preferably be light-weight and easily stored in a work truck or shop to be transported to jobs as needed. The air pump 25 may include a rechargeable power source 70. This is necessary so that a user may employ the device 10 in areas with no available external power source 15. For example, the device 10 may be used in construction sites that are not yet provided with a ready supply of electricity. The rechargeable power source 70 may be a rechargeable battery as is obvious to one ordinarily skilled in the art and may further be recharged by connection to an external power source 15, such as a wall outlet, for example.

Additionally, the automatic ambient air receiving and distributing mechanism 21 may include a portable air intake manifold 26 communicatively coupled to the air-pump 25 for receiving and distributing the ambient air 12 based upon the pre-defined operating parameters. Further, the ambient air may be directed into the air intake manifold 26 at an ambient air intake rate 22 and thereafter distributed away from the air intake manifold 26 at ambient air distribution rates 23 respectively.

The air receiving and distributing mechanism 21 may also include each of the ambient air distribution rates 23 traveling along mutually exclusive paths 27 away from the air intake manifold 26. The portable air pump 25 is beneficial wherein a user may place the portable air pump 25 in an area containing ambient air 12 with a lower temperature, such as inside an air-conditioning space or under a shaded tree, for example. In this manner, cool air 12 is distributed to the user even if the user is mobile or is working in a hot exterior areas.

Referring to FIGS. 3A-5, the automatic ambient air receiving and distributing mechanism 21 may further include a user interface 28 and a controller 29 communicatively coupled to the user interface 28. The controller 29 may also be coupled with a rechargeable power source 71 such as a battery, as is obvious to one skilled in the art. The rechargeable power source 71 may be recharged by connection to an external power source 15, such as a wall outlet, for example. In this manner, the user may transport the air intake manifold 26 and controller 29 to an area remote from the air pump 25. This enables the air pump 25 to remain in an area with cooler ambient air such as an air-conditioned space or under shade, although the user is in an outside area with hot ambient air.

In addition, the mechanism preferably includes an intake valve 30 positioned upstream of the controller 29 for receiving the ambient air 12 at the ambient air intake rate 22. Also, the mechanism 21 may include an intake valve sensor 31 communicatively coupled to the intake valve 30 that is preferably situated downstream thereof. Such an intake valve sensor 31 preferably generates and transmits a main signal 32 to the controller 29 that identifies a level of the ambient air intake rate 22. A plurality of distribution valves 33 positioned downstream of the controller 29 for receiving the ambient air 12 at the distribution rates 23 respectively is also preferably included.

Further, the mechanism 21 may include a plurality of distribution valve sensors 34 communicatively coupled to the distribution valves 33 that may be situated downstream thereof respectively. Each of such distribution valve sensors 34 preferably generates and transmits auxiliary signals 35 to the controller 29 that identify a corresponding level of the ambient air distribution rates 23 respectively. This is vital so that the device 10 may correctly adjust to attain the proper intake rate 22 of air when multiple users are in fluid communication with the air intake manifold 26. Additionally, the mechanism 21 may include a plurality of conduits 36 connected to the intake valve 30 and the distribution valves 33 respectively. Each of such conduits 36 travels along mutually exclusive paths and further may be arranged in such a manner that a separate and unique volume of the ambient air is preferably directed through the conduits 36 during operating conditions. The combined elements of the separate conduits provide the unexpected benefit wherein multiple users may be coupled to a single portable air pump 25. This feature will save money as one piece of equipment may support multiple users with alternating air supplies.

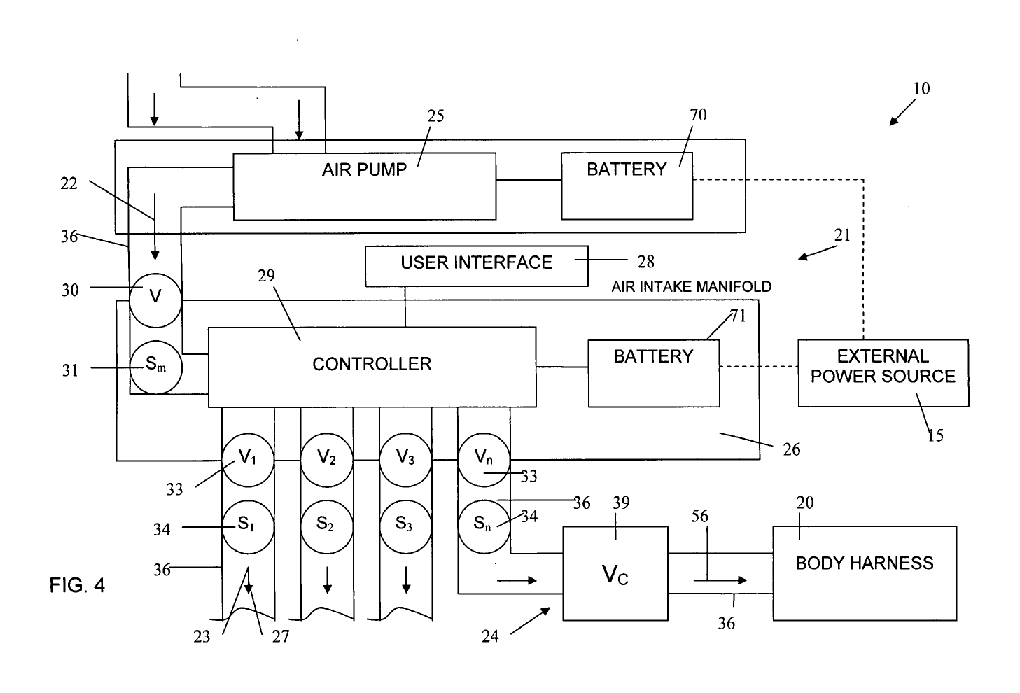

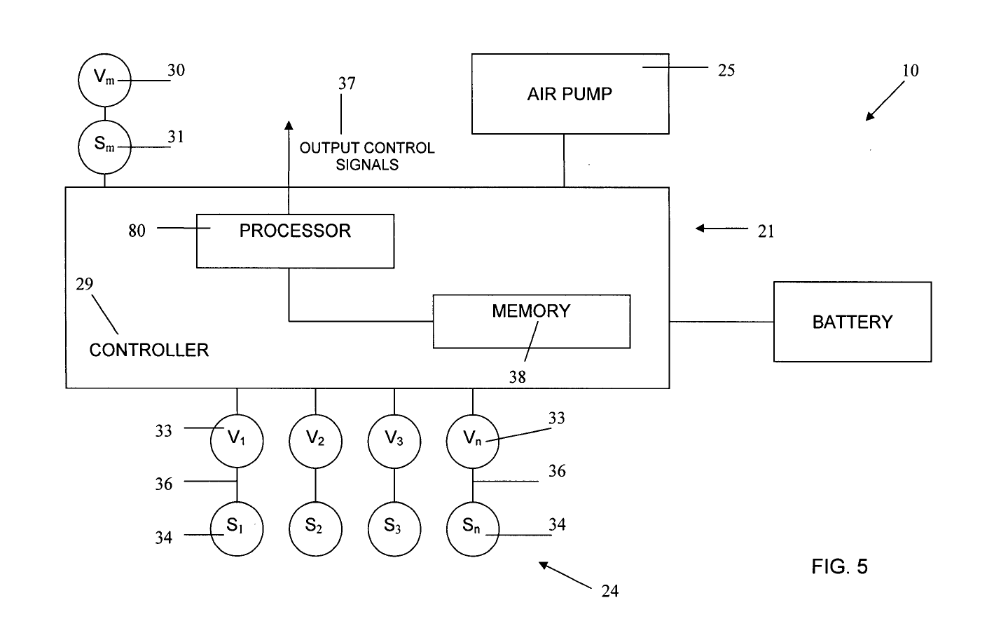

Referring to FIGS. 4 and 5, the body temperature regulating device 10 may further include a memory 38 electronically coupled with a processor 80. Such a memory 38 may include programmable software instructions that cause the distribution valves 33 to automatically toggle between open and closed positions based upon real-time fluctuations detected in the operating parameters. The software instructions may further include a programmable control logic algorithm preferably executing the steps of first requesting a user to designate a desired level of the ambient air intake rate 22 via the user interface 28.

Secondly, the algorithm preferably requests a user to designate a desired level of the ambient air distribution rates 23 via the user interface 28. Third, the algorithm may receive the main signal 32 and learn a real-time ambient air intake rate 22 therefrom. Next, the algorithm may receive the auxiliary signals and learn real-time ambient air distribution rates 23 therefrom respectively.

A fourth step preferably includes determining whether one of the real-time ambient air intake 22 and distribution 23 rates does not equal a corresponding one of the desired ambient air intake 22 and distribution 23 rates levels respectively. If the fourth step is yes, then the algorithm's fifth step may include generating and transmitting a corresponding output control signal 37 to an associated one of the intake 30 and distribution 33 valves to thereby automatically adjust the one real-time ambient air rate to a corresponding one of the desired ambient air intake 22 and distribution 23 rates respectively. This feature is especially beneficial by providing a higher intake rate 22 when multiples users are attached to the ambient air exhaust rate adjusting mechanism 24.

Referring to FIGS. 3A-5, the ambient air exhaust rate adjusting mechanism 24 may include a comfort valve 39 located downstream of an associated one of the conduits 36 and located proximate to the body harness 20. Such a comfort valve 39 may include a hollow chamber 40 and an inlet port 41 located at a first quadrant 42 of the hollow chamber 40 that may be in fluid communication with the one conduit 36 for receiving the ambient air 12 therein. The comfort valve 39 may additionally include an outlet port 43 located at a second quadrant 44 of the hollow chamber 40 that may be in fluid communication with the body harness 20.

Further, the comfort valve 39 may include a spool 45 centrally seated within the hollow chamber 39 that may be freely rotated along a unidirectional arcuate path defined within the hollow chamber 40. In addition, a curvilinear first passageway 47 may be defined about the spool 45 and travel within the hollow chamber 40 of the comfort valve 39. The comfort valve 39 may also include an actuating block 48 statically nested within the hollow chamber 40 that preferably maintains intermittent engagement with the spool 45 as the spool is rotated. The combined elements of the comfort valve 39 being located proximate to the body harness 20 on each of the conduits 36 provide an unexpected benefit wherein a user may remotely adjust the rate of ambient air flowing into the user's body harness 20. In this manner, each user may make individual adjustments without interrupting any task at hand or disturbing the air exhaust rates of other users.

Referring to FIGS. 3A-5, the ambient air exhaust rate adjusting mechanism 24 further may include diametrically opposed orifices 49 formed within an outer wall 50 of the spool 45 such that a linear second passageway 51 may be formed through a center 52 of the spool 45. Also, the mechanism 24 may include first 53 and second 54 arms pivotally connected to the spool 45 and radially extending out therefrom. Such first 53 and second 54 arms preferably maintain direct abutment with an inner surface 55 of the hollow chamber 40 when articulated to open positions. This operates so that the ambient air 12 is prohibited from traveling beyond the first 53 and second 54 arms in the curvilinear first 47 passageway.

The spool 45 may be manually rotated by the user to thereby cause one of the first 53 and second 54 arms to rub against the actuating block 48. One arm 54 may be pivotally positioned over a respective one of the orifices 49 to thereby axially offset the linear second 51 passageway away from the outlet port 43. This operates so that the ambient air 12 is diverted away from the linear second 51 passageway and along the curvilinear first 47 passageway.

Further, the ambient air exhaust rate 56 is at maximum and minimum levels when the linear second 51 passageway is axially and transversely aligned with the outlet port 43 respectively. The combined elements of the first and second passageways and the comfort valve provide a benefit, wherein a user may adjust their individual air exhaust rate or even suspend their air exhaust altogether. This is useful in a situation where each user must disengage the conduit while conserving cool air for other users attached to the ambient air receiving and distributing mechanism 21.

In use, a method for distributing ambient air directly onto a user skin preferably includes the chronological step of first providing and positioning a body harness 20 directly on the user skin 11. The method may include the second step of automatically receiving and distributing ambient air towards the body harness 20 based upon a plurality of pre-defined operating parameters.

The pre-defined operating parameters may include an ambient air intake rate 22 measured upstream of the body harness. The parameters may also include a plurality of ambient air distribution rates 23 measured downstream of the ambient air intake rate 22. Finally, the method may include the third step of manually adjusting an ambient air exhaust rate 56 leading directly into the body harness 20 to thereby manually and independently override the pre-defined operating parameters when desired by the user. The ambient air exhaust rate 56 is preferably located downstream of the ambient air distribution rates 23.

While the invention has been described with respect to a certain specific embodiment, it will be appreciated that many modifications and changes may be made by those skilled in the art without departing from the spirit of the invention. It is intended, therefore, by the appended claims to cover all such modifications and changes as fall within the true spirit and scope of the invention.

In particular, with respect to the above description, it is to be realized that the optimum dimensional relationships for the parts of the present invention may include variations in size, materials, shape, form, function and manner of operation. The assembly and use of the present invention are deemed readily apparent and obvious to one skilled in the art.

What is claimed as new and what is desired to secure by Letters Patent of the United States is:

1. A body temperature regulating device for distributing ambient air directly onto a user skin, said body temperature regulating device comprising:
a body harness adapted to be directly positioned on the user skin;
means for automatically receiving and distributing ambient air towards said body harness based upon a plurality of pre-defined operating parameters, said pre-defined operating parameters comprising
an ambient air intake rate measured upstream of said body harness, and
a plurality of ambient air distribution rates measured downstream of said ambient air intake rate; and
means for manually adjusting an ambient air exhaust rate leading directly into said body harness to thereby manually and independently override said pre-defined operating parameters when desired by the user;
wherein said ambient air exhaust rate is located downstream of said ambient air distribution rates.

2. The body temperature regulating device of claim 1, wherein said automatic ambient air receiving and distributing means comprises:
a portable air-pump for receiving and channeling the ambient air; and
a portable air intake manifold communicatively coupled to said air-pump for receiving and distributing the ambient air based upon said pre-defined operating parameters;
wherein the ambient air is directed into said air intake manifold at an ambient air intake rate and thereafter distributed away from said air intake manifold at ambient air distribution rates respectively;
wherein each of said ambient air distribution rates travel along mutually exclusive paths away from said air intake manifold.

3. The body temperature regulating device of claim 2, wherein said automatic ambient air receiving and distributing means further comprises:
a user interface;
a controller communicatively coupled to said user interface;
an intake valve positioned upstream of said controller for receiving the ambient air at said ambient air intake rate;
an intake valve sensor communicatively coupled to said intake valve and being situated downstream thereof, said intake valve sensor generating and transmitting a main signal to said controller that identifies a level of said ambient air intake rate;
a plurality of distribution valves positioned downstream of said controller for receiving the ambient air at said distribution rates respectively;
a plurality of distribution valve sensors communicatively coupled to said distribution valves and being situated downstream thereof respectively, each of said distribution valve sensors generating and transmitting auxiliary signals to said controller that identify a corresponding level of said ambient air distribution rates respectively; and
a plurality of conduits connected to said intake valve and said distribution valves respectively, each of said conduits traveling along mutually exclusive paths and further being arranged in such a manner that a separate and unique volume of the ambient air is directed through said conduits during operating conditions.

4. The body temperature regulating device of claim 3, wherein said memory comprises: programmable software instructions that causes said distribution valves to automatically toggle between open and closed positions based upon real-time fluctuations detected in said operating parameters, said software instructions including a programmable control logic algorithm executing the steps of
a. requesting a user to designate a desired level of said ambient air intake rate via said user interface;
b. requesting a user to designate a desired level of said ambient air distribution rates via said user interface;
c. receiving said main signal and learning a real-time ambient air intake rate therefrom;
d. receiving said auxiliary signals and learning real-time ambient air distribution rates therefrom respectively;
e. determining whether one of said real-time ambient air intake and distribution rates do not equal a corresponding one of said desired ambient air intake and distribution rates levels respectively; and
f. if yes, then generating and transmitting a corresponding output control signal to an associated one of said intake and distribution valves to thereby automatically adjust said one real-time ambient air rate to a corresponding one of said desired ambient air intake and distribution rates respectively.

5. The body temperature regulating device of claim 4, wherein said ambient air exhaust rate adjusting means comprises:
a comfort valve located downstream of an associated one of said conduits and located proximate to said body harness, said comfort valve including
a hollow chamber,
an inlet port located at a first quadrant of said hollow chamber and being in fluid communication with said one conduit for receiving the ambient air therein,
an outlet port located at a second quadrant of said hollow chamber and being in fluid communication with said body harness,
a spool centrally seated within said hollow chamber and being freely rotated along a unidirectional arcuate path defined within said hollow chamber,
a curvilinear first passageway defined about said spool and traveling within said hollow chamber, and
an actuating block statically nested within said hollow chamber and maintaining intermittent engagement with said spool as spool is rotated.

6. The body temperature regulating device of claim 5, wherein said ambient air exhaust rate adjusting means further comprises:
diametrically opposed orifices formed within an outer wall of said spool such that a linear second passageway is formed through a center of said spool; and
first and second arms pivotally connected to said spool and radially extending out therefrom, said first and second arms maintaining direct abutment with an inner surface of said hollow chamber when articulated to an open positions such that the ambient air is prohibited from traveling beyond said first and second arms in said curvilinear first passageway;
wherein said spool is manually rotated by the user and thereby causes one of said first and second arms to rub against said actuating block, said one arm being pivotally positioned over a respective one of said orifices and thereby axially offsetting said linear second passageway away from said outlet port such that the ambient air is diverted away from said linear second passageway and along said curvilinear first passageway.

7. The body temperature regulating device of claim 6, wherein said ambient air exhaust rate is at maximum and minimum levels when said linear second passageway is axially and transversely aligned with said outlet port respectively.

8. A body temperature regulating device for distributing ambient air directly onto a user skin, said body temperature regulating device comprising:
a body harness adapted to be directly positioned on the user skin;
means for automatically receiving and distributing ambient air towards said body harness based upon a plurality of pre-defined operating parameters, said pre-defined operating parameters comprising
an ambient air intake rate measured upstream of said body harness, and
a plurality of ambient air distribution rates measured downstream of said ambient air intake rate; and
means for manually adjusting an ambient air exhaust rate leading directly into said body harness to thereby manually and independently override said pre-defined operating parameters when desired by the user.

9. The body temperature regulating device of claim 8, wherein said automatic ambient air receiving and distributing means comprises:
a portable air-pump for receiving and channeling the ambient air; and
a portable air intake manifold communicatively coupled to said air-pump for receiving and distributing the ambient air based upon said pre-defined operating parameters;
wherein the ambient air is directed into said air intake manifold at an ambient air intake rate and thereafter distributed away from said air intake manifold at ambient air distribution rates respectively;
wherein each of said ambient air distribution rates travel along mutually exclusive paths away from said air intake manifold.

10. The body temperature regulating device of claim 9, wherein said automatic ambient air receiving and distributing means further comprises:
a user interface;
a controller communicatively coupled to said user interface;
an intake valve positioned upstream of said controller for receiving the ambient air at said ambient air intake rate;
an intake valve sensor communicatively coupled to said intake valve and being situated downstream thereof, said intake valve sensor generating and transmitting a main signal to said controller that identifies a level of said ambient air intake rate;
a plurality of distribution valves positioned downstream of said controller for receiving the ambient air at said distribution rates respectively;
a plurality of distribution valve sensors communicatively coupled to said distribution valves and being situated downstream thereof respectively, each of said distribution valve sensors generating and transmitting auxiliary signals to said controller that identify a corresponding level of said ambient air distribution rates respectively; and
a plurality of conduits connected to said intake valve and said distribution valves respectively, each of said conduits traveling along mutually exclusive paths and further being arranged in such a manner that a separate and unique volume of the ambient air is directed through said conduits during operating conditions.

11. The body temperature regulating device of claim 10, wherein said memory comprises: programmable software instructions that causes said distribution valves to automatically toggle between open and closed positions based upon real-time fluctuations detected in said operating parameters, said software instructions including a programmable control logic algorithm executing the steps of
a. requesting a user to designate a desired level of said ambient air intake rate via said user interface;
b. requesting a user to designate a desired level of said ambient air distribution rates via said user interface;
c. receiving said main signal and learning a real-time ambient air intake rate therefrom;
d. receiving said auxiliary signals and learning real-time ambient air distribution rates therefrom respectively;
e. determining whether one of said real-time ambient air intake and distribution rates do not equal a corresponding one of said desired ambient air intake and distribution rates levels respectively; and
f. if yes, then generating and transmitting a corresponding output control signal to an associated one of said intake and distribution valves to thereby automatically adjust said one real-time ambient air rate to a corresponding one of said desired ambient air intake and distribution rates respectively.

12. The body temperature regulating device of claim 11, wherein said ambient air exhaust rate adjusting means comprises:
a comfort valve located downstream of an associated one of said conduits and located proximate to said body harness, said comfort valve including
a hollow chamber,
an inlet port located at a first quadrant of said hollow chamber and being in fluid communication with said one conduit for receiving the ambient air therein,
an outlet port located at a second quadrant of said hollow chamber and being in fluid communication with said body harness,
a spool centrally seated within said hollow chamber and being freely rotated along a unidirectional arcuate path defined within said hollow chamber,
a curvilinear first passageway defined about said spool and traveling within said hollow chamber, and
an actuating block statically nested within said hollow chamber and maintaining intermittent engagement with said spool as spool is rotated.

13. The body temperature regulating device of claim 12, wherein said ambient air exhaust rate adjusting means further comprises:
diametrically opposed orifices formed within an outer wall of said spool such that a linear second passageway is formed through a center of said spool; and
first and second arms pivotally connected to said spool and radially extending out therefrom, said first and second arms maintaining direct abutment with an inner surface of said hollow chamber when articulated to an open positions such that the ambient air is prohibited from traveling beyond said first and second arms in said curvilinear first passageway;
wherein said spool is manually rotated by the user and thereby causes one of said first and second arms to rub against said actuating block, said one arm being pivotally positioned over a respective one of said orifices and thereby axially offsetting said linear second passageway away from said outlet port such that the ambient air is diverted away from said linear second passageway and along said curvilinear first passageway.

14. The body temperature regulating device of claim 13, wherein said ambient air exhaust rate is at maximum and minimum levels when said linear second passageway is axially and transversely aligned with said outlet port respectively.

15. A method for distributing ambient air directly onto a user skin, said method comprising the chronological steps of:
a. providing and positioning a body harness directly on the user skin;
b. automatically receiving and distributing ambient air towards said body harness based upon a plurality of pre-defined operating parameters, said pre-defined operating parameters comprising an ambient air intake rate measured upstream of said body harness, and a plurality of ambient air distribution rates measured downstream of said ambient air intake rate; and
c. manually adjusting an ambient air exhaust rate leading directly into said body harness to thereby manually and independently override said pre-defined operating parameters when desired by the user, wherein said ambient air exhaust rate is located downstream of said ambient air distribution rates.
